(19) United States
(12) Patent Application Publication
(10) Pub. No.: US 2018/0000870 A1
(43) Pub. Date: Jan. 4, 2018

(54) METHODS OF FORMING AMNIOTIC FLUID-DERIVED PREPARATIONS

(71) Applicant: Applied Biologics, LLC, Scottsdale, AZ (US)

(72) Inventor: Edward Britt, Scottsdale, AZ (US)

(21) Appl. No.: 15/637,945

(22) Filed: Jun. 29, 2017

Related U.S. Application Data
(60) Provisional application No. 62/356,299, filed on Jun. 29, 2016.

(51) Int. Cl. A61K 35/50; A61K 9/19

(52) U.S. Cl. CPC A61K 35/50; A61K 9/19

(57) ABSTRACT

The invention relates to methods of forming derivatives of amniotic fluid. Specifically, embodiments of the disclosed invention relate to methods of forming amniotic fluid-derived preparations from donor amniotic fluid for use in clinical medicine and medical research.

### CROSS-REFERENCE TO RELATED APPLICATIONS

This application claims the benefit of and priority to U.S. Provisional Patent Application No. 62/356,299, filed Jun. 29, 2016, the entire contents of which are incorporated herein by reference.

### BACKGROUND OF THE INVENTION

### Technical Field

This invention relates to methods of forming amniotic fluid preparations; in particular, embodiments of the invention relate to methods for forming amniotic fluid preparations by combining proteins and cells derived from donor amniotic fluid with non-biologically derived fluids for use in therapeutic and research applications.

### State of the Art

Amniotic fluid, specifically human amniotic fluid, has been identified as a rich source of therapeutic biomolecules. Amniotic fluid's suspended protein fraction and cellular components contain a complex biologic soup of growth factors, inflammatory mediators, immuno-modulators, and other active biomolecules. Amniotic fluid is rich in pluripotent cellular elements, including stem cells, which both contain and secrete relatively high concentrations of these and other biologically active substances into the extracellular milieu.

Amniotic fluid derivatives, with or without a cellular component, have tremendous potential for use in a range of medical applications. An amniotic fluid-derived product, however, must be processed and concentrated in a manner which preserves protein bioactivity and cellular viability, quantified with respect to protein and cellular components, and then packaged for convenient and practical use by a clinician or researcher. Without a standardized amniotic fluid-derived product, clinical use—including clinical trials in human subjects—may be unpredictable and/or unsafe. Further, it will be impossible for researchers to obtain reproducible results in research applications. Currently available methods for preparation of amniotic fluid derivatives specific to certain target functions, clinical therapies, and investigational use have not been described.

Accordingly, what are needed are methods to produce amniotic fluid-derived products which are commercially available, of consistent quality, and safe for clinical and investigational use.

Citation of documents herein is not an admission by the applicant that any is pertinent prior art. Stated dates or representation of the contents of any document is based on the information available to the applicant and does not constitute any admission of the correctness of the dates or contents of any document.

### SUMMARY OF THE INVENTION

Disclosed are methods of forming amniotic fluid-derived preparations.

Disclosed is a method of forming an amniotic fluid-derived preparation comprising the steps of separating a donor amniotic fluid into a supernatant and a cellular component; and combining the supernatant, the cellular component; and a fluid into an amniotic fluid-derived preparation.

In some embodiments, the separating step comprises centrifuging the donor amniotic fluid. In some embodiments, the separating step comprises filtering the donor amniotic fluid.

In some embodiments, the fluid comprises a cryopreservative. In some embodiments, the fluid comprises a buffered electrolyte solution.

In some embodiments, the method further comprises a step selecting a donor amniotic fluid. In some embodiments, the selecting step comprises determining a risk of a transmittable infectious disease in the donor amniotic fluid.

In some embodiments, the method further comprises a step collecting a donor amniotic fluid. In some embodiments, the collecting step comprises removal of the donor amniotic fluid from a pregnant uterus by Cesarean section. In some embodiments, the collecting step comprises trans-vaginal removal of the donor amniotic fluid.

In some embodiments, the method further comprises a step processing a donor amniotic fluid. In some embodiments, the method further comprises a step standardizing a biologic activity of the supernatant. In some embodiments, the method further comprises a step standardizing a cell count of the cellular component. In some embodiments, the method further comprises a step packaging the amniotic fluid-derived preparation.

Disclosed is a method of forming an amniotic fluid-derived preparation comprising the steps of separating a donor amniotic fluid into a supernatant and a cellular component; and combining the supernatant and a fluid into an amniotic fluid-derived preparation.

In some embodiments, the method further comprises a step lyophilizing the amniotic fluid-derived preparation.

Disclosed is a method of forming an amniotic fluid-derived preparation comprising the steps of separating a donor amniotic fluid into a supernatant and a cellular component; and combining the cellular component and a fluid into an amniotic fluid-derived preparation.

In some embodiments, the fluid comprises a cryopreservative. In some embodiments, the fluid comprises a buffered electrolyte solution.

In some embodiments, the donor amniotic fluid supernatant is acellular (i.e., the acellular supernatant is entirely free of cells). In some embodiments, the supernatant is nearly entirely free of cells, for example, the supernatant contains fewer than 10,000 cells/ml, fewer than 1,000 cells/ml, fewer than 100 cells/ml, or fewer than 10 cells/ml.

The foregoing and other features and advantages of the present invention will be apparent from the following more detailed description of the particular embodiments of the invention, as illustrated in the accompanying drawings.

### BRIEF DESCRIPTION OF THE DRAWINGS

FIG. 1 is a flow chart representing a method 100 of forming an amniotic fluid preparation;

FIG. 2 is a flow chart representing a further embodiment of method 100 of forming an amniotic fluid preparation;

FIG. 3 is a flow chart representing a further embodiment of method 100 of forming an amniotic fluid preparation;

FIG. 4 is a flow chart representing a further embodiment of method 100 of forming an amniotic fluid preparation;

FIG. 5 is a flow chart representing a further embodiment of method 100 of forming an amniotic fluid preparation;

FIG. 6 is a flow chart representing a further embodiment of method 100 of forming an amniotic fluid preparation;

FIG. 7 is a flow chart representing a further embodiment of method 100 of forming an amniotic fluid preparation;

FIG. 8 is a flow chart representing a method 200 of forming an amniotic fluid preparation;

FIG. 9 is a flow chart representing a further embodiment of method 200 of forming an amniotic fluid preparation; and

FIG. 10 is a flow chart representing a method 300 of forming an amniotic fluid preparation.

### DETAILED DESCRIPTION OF THE INVENTION

A detailed description of the hereinafter disclosed methods and apparatus associated therewith are presented herein by way of exemplification and not limitation with reference to the Figures. Although certain embodiments are shown and described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure. As a preface to the detailed description, it should be noted that, as used in this specification and the appended claims, the singular forms “a”, “an” and “the” include plural referents, unless the context clearly dictates otherwise.

The disclosed invention relates to methods of forming amniotic fluid-derived preparations. Specifically, embodiments of the invention comprise methods of forming preparations from cellular and non-cellular derivatives of amniotic fluid. The disclosed embodiments of methods of forming amniotic fluid-derived preparations will be used in tissue regenerative therapy, treatment of degenerative disease, treatment of other clinical conditions, and research into the treatment of multiple surgical and non-surgical injuries and disease states.

It is to be understood that some of the terms used herein to disclose the elements and various embodiments of the present invention may have a broad meaning according to at least the definitions provided herein below.

“Amniotic fluid” means fluid originating in the amniotic sac of a pregnant female and comprising suspended cellular and non-cellular elements, including all defined and undefined components, molecules, and compounds. This fluid, which derives from both maternal plasma and the developing embryo and fetus, comprises water, electrolytes, proteins and other classes of biologically active molecules, and cells. The cellular component includes epithelial and mesenchymal stem cells of both fetal and maternal origin.

“Donor amniotic fluid” (“donor AF”) means amniotic fluid (“AF”) utilized to form an amniotic fluid-derived preparation. “Donor” is used herein to refer to AF collected from a single pregnant mammal, including a pregnant human. Donor is also used to refer to the pregnant mammal or human herself. Whether “donor” means the AF or the pregnant female will be clear from the context. Donor AF is singular or plural, meaning AF from a single donor of AF from a plurality of donors, and is specifically referred to herein as “a donor AF,” “multiple donor AFs,” for example.

“Preparation” means a substance specially made up from constituent substances. For example, an amniotic fluid-derived preparation is a substance specially made up from at least one or more of a group of constituent substances comprising a cellular component, a supernatant, and a fluid.

“Cellular component” means intact cells originating in amniotic fluid. Generally, the cellular component is suspended within the fluid component of AF. The cellular component includes any cell type, whether defined and known or undefined and unknown, which may be present in AF. “Stem cells” means undifferentiated cells which may give rise to additional generations of stem cells or which may differentiate into progenitor cells. When used in this application, “stem cell” means a stem cell originating in the cellular component of AF, however, stem cells may otherwise originate in fetal membranes, umbilical cord blood, or non-fetal tissues. “Epithelial stem cell” means a stem cell originating from the embryonic epithelium, including the ectoderm and the endoderm embryonic layers. “Mesenchymal stem cell” means a stem cell originating from the embryonic mesenchyme, including stromal and vascular tissue of the umbilical cord. Wherein “stem cell” is used as referring to a stem cell not originating in the cellular component of AF, this will be explicitly noted in the specification. “Progenitor cell” means a cell which is committed to differentiating 1) along a specific germ cell line, i.e. ectoderm, mesoderm, or endoderm; and 2) a cell committed to differentiating into a specific cell or tissue, i.e. chondrocyte or integrated cortical columnar unit.

“Centrifugation” means a process wherein centrifugal force is used to separate materials of different densities, including cells, organelles, and cellular debris; exosomes; solutions; large molecules, such as proteins or nucleic acids; colloidal particles suspended in a liquid, and the like. “Relative centrifugal force” means the radial force generated by a spinning centrifuge rotor expressed relative to the earth's gravitational force. For example, a relative centrifugal force of 100 g means a radial force one hundred (100) times the force of gravity.

“Precipitation” means a reaction wherein cations and anions in an aqueous solution combine to form an insoluble ionic solid.

“Filtration” means a process wherein solids suspended in a fluid are physically separated from the fluid by passing the suspension through a medium through which only the fluid can pass. The solids to be physically separated from the fluid include (but are not limited to) viable cells, dead cells, cellular debris, organelles, exosomes, precipitates including proteins and nucleic acids, and the like. “Ultrafiltration” means filtration utilizing a filtration medium comprising a semipermeable membrane wherein separation is augmented by a force, such as hydrostatic pressure or osmotic pressure.

“Supernatant” means the fluid which may be removed from a centrifuge tube or other container containing a solid or semisolid pellet at the end of a centrifuge tube following centrifugation, such as by pipetting or decanting. The meaning of “supernatant” additionally includes any fluid covering a solid residue following crystallization, precipitation, or other process causing the solid residue to become distinct from the covering fluid. Supernatant includes water or other liquid and all constituent materials, including compounds in solution or suspension and intact cells, cellular elements, organelles, membrane fragments, and the like remaining in suspension following centrifugation, precipitation, and the like.

“Protein fraction” means at least one protein comprising AF. Protein fraction is a portion of an AF supernatant containing a protein. A protein fraction may contain one protein or the entire amniotic fluid proteome. A protein fraction may comprise an entire AF supernatant or any portion of an AF supernatant comprising at least one protein arising from AF. A protein fraction may comprise an additional non-AF protein from a secondary source separate from a donor AF, including an AF protein from a second donor AF, a non-AF protein, or an AF or other protein produced outside of AF by other means such as by a genetically engineered bacterium, extracellular in vitro protein synthesis, and the like.

“K_{d}” means a dissociation constant, such as the dissociation constant of an enzyme, an antibody, and the like. “Buffer solution” means an aqueous solution comprising a weak acid and its conjugate base used to stabilize the pH by resisting changes in pH when acid or base is added. A buffer solution is used to stabilize the pH of the solution within a narrow range around a specific value. “Buffer solution” is used generically herein to mean any buffer solution appropriate for a given application and not one specific buffer solution. Examples of suitable buffer solutions include a phosphate buffer solution (“PBS”) and buffer solutions commonly used in biologic applications.

“Donor” means a pregnant female, including a peripartum female delivering an infant, from whom amniotic fluid is obtained. “Fetal placental membranes” is used synonymously with “fetal membranes” and means any or all of the amnion, chorion, and Wharton's jelly. “Lyophilization” means drying by removal of water through sublimation of water ice directly to water vapor without passing through a liquid phase. “Concentrated” means a relative concentration of a cell, a protein, a non-cellular non-protein substance or other material per unit volume that is greater than the original concentration of that substance in the donor AF. “Substantially depleted” means a concentration of a cell, a protein, a non-cellular non-protein substance, or other material per unit volume of a preparation or fluid wherein the concentration is less than ten (10) percent of the concentration of that material in a donor AF. Where “substantially depleted” means a specific concentration, this is indicated herein.

“Immunoglobulin” means any one or more specific proteins belonging to the family of proteins which may be produced by white blood cells and acting as antibodies. “Albumin” means a soluble, monomeric human protein, United States National Center for Biotechnology Information (“NCBI”) accession number CAA00606.1. “Lysate” means the intracellular products released by the disruption of a cell membrane by mechanical or chemical means.

The disclosed invention relates to amniotic fluid-derived preparations. Specifically, embodiments of the invention comprise preparations formed from cellular and non-cellular derivatives of amniotic fluid. The disclosed embodiments of amniotic fluid-derived preparations may be used in tissue regenerative therapy, other medical therapies, and research into the treatment of multiple surgical and non-surgical degenerative conditions.

AF and its constituent components occupy a unique position in the field of regenerative medicine. The fluid, which derives from both maternal plasma and the developing embryo and fetus, comprises water, electrolytes, proteins and other classes of biologically active molecules, and cells. The cellular component includes epithelial and mesenchymal stem cells of both fetal and maternal origin.

AF may be separated into a cellular component and a supernatant by centrifugation. The cellular component includes different families of stem cells, of both embryonic and extra-embryonic (maternal) origin. AF stem cells include both epithelial and mesenchymal stem cells. These stem cells are often capable of engraftment and differentiation within host tissue of another individual. Additionally, amniotic fluid stem cells neither express human leukocyte class I antigens (“HLA-1”) nor have immune-competence associated with non-embryonic hematopoietic stem cells. Consequently, transplanted amniocytes do not provoke an immune response in the recipient and do not differentiate into host-sensitized T-lymphocytes capable of mounting a graft-versus-host reaction.

The supernatant contains a large variety and concentration of proteins. In addition to albumin and immunoglobulin, multiple families of regulatory proteins are present which likely affect fetal growth, development, and interaction with the maternal physiologic environment. Growth factors secreted by the mother and fetus are the principal non-cellular active biological compounds native to amniotic fluid. Systematic evaluation of the human amniotic fluid proteome has identified numerous proteins within gene ontology (“GO”) categories relevant to tissue healing, regenerative bioactivity, and biologic augmentation. GO categories are functional identifiers of gene and protein networks that indicate the biologically significant proteins naturally present in amniotic fluid. Key GO categories that have so far been identified include 1) cellular movement; 2) development and function; 3) cellular growth and proliferation; 4) cell-to-cell signaling and interaction; 5) tissue differentiation; and 6) organism development. These gene ontology-classifiers identify the presence of specific categories of growth factors and growth factor networks directly associated with regenerative bioactivity (Cho, et al., (2012) “Proteomic analysis of human amniotic fluid” Mol Cell Proteomics 6:1406-15).

Distinguishing and separating protein families comprising distinct GO categories allows for supplementation of an amniotic fluid-derived preparation with proteins from a distinct GO category to achieve the desired therapeutic effect. For example, proteins associated with the cellular growth and proliferation GO category can be isolated, concentrated, and then added to an amniotic fluid-derived preparation for treatment of a non-healing wound. In an additional example, isolated and concentrated proteins associated with the tissue differentiation GO category can supplant an amniotic fluid-derived preparation used therapeutically to induce stem cells present in the preparation, following injection of the preparation into a knee joint with a damaged meniscus, to differentiate into chondrocytes.

Unlike fetal placental membranes, it is generally not practical to obtain AF from a donor planning a vaginal delivery because in the majority of vaginal deliveries the placental membranes spontaneously rupture and the AF is lost. Controlled, therapeutic rupture of membranes, however, is an exception and is discussed herein below. The use of AF from donors undergoing a Cesarean-section delivery essentially eliminates gross bacterial contamination of the donor AF. Of the approximately 4 million births annually in the U.S. mentioned earlier, approximately 33%—1.32 million overall—are by Cesarean delivery which reduces the potential donor pool for AF by nearly seventy percent. AF, therefore, is potentially available to develop derived preparations from a total between 0.95 and 1.32 million births annually in the U.S.

As mentioned earlier, AF may be collected from suitable volunteer donors and processed for storage prior to forming amniotic fluid-derived preparations for use in a variety of surgical procedures and non-surgical clinical applications. Some non-limiting examples of non-surgical clinical applications include use of amniotic fluid-derived preparations in dressings and wound treatments as an adjunct to healing, particularly in the treatment of chronically ischemic or infected wounds; as a component in the creation of artificial skin, and to augment healing of tendon and ligamentous injuries. Surgical uses of an amniotic fluid-derived preparation include introduction as an adjunct to healing of surgically repaired bone, tendon, other soft tissue, and open wounds; a means to mitigate the formation of scar tissue and adhesions, and other beneficial applications in surgery and non-surgical minimally invasive medical therapies. Amniotic fluid-derived preparation may be added to augment biologic dressings, which are commercially available from a variety of sources, with stem cells and growth factors to treat burns, skin pressure ulcers, other chronic open wounds, corneal ulcers, and as a dressing following corneal transplant and other ocular procedures. Amniotic fluid-derived preparation may be used as a component of the extracellular matrix in bioengineered connective tissue scaffolding for tissue regeneration and organogenesis using extraembryonic stem cells and other progenitor cells. Amniotic fluid-derived preparation possesses the anti-inflammatory properties of AF and may be used to prevent the development of postoperative adhesions between the tendon, tendon sheath, and associated tissue following tenolysis, synoviolysis, surgical repair of a damaged tendon, and surgical debridement of necrotic or damaged tendon tissue. Amniotic fluid-derived preparation may also be useful to mitigate nerve cell death and promote axonal regeneration following early repair of peripheral nerve transections.

An injectable amniotic fluid-derived preparation allows for use of the product in both surgical and minimally invasive settings. The injectable amniotic fluid-derived preparation may be injected into a defined closed space near the end of the surgical procedure, but prior to closing superficial layers of muscle, fascia, and skin at a time when precise placement of the preparation under the surgeon's direct visualization is possible. For example, an injectable amniotic fluid-derived preparation, depending on the viscosity of the final product, is delivered by injection though a hypodermic needle as small as 30-gauge (“G”) into a closed tendon sheath following tenolysis or tendon repair, into a closed joint capsule following repair of intra-articular cartilage, ligaments, or total joint replacement, into the peritoneal cavity following closure of the abdominal wall, into the pleural space following closure of the chest wall, and into the subdural space following closure of the spinal or intracranial dura mater. An injectable amniotic fluid-derived preparation of higher viscosity is injected through a 23 G, 22 G, 21 G, 20 G, 18 G, 16 G, or larger-bore hypodermic needle in these and other surgical and minimally invasive applications. An injectable amniotic fluid-derived preparation of lower viscosity is injected through a 25 G or 30 G needle for use in fine neural repair, aesthetic surgery, and other applications. Following wound closure, an injectable amniotic fluid-derived preparation may also be re-injected into the defined closed space during the perioperative and postoperative period if deemed useful by the surgeon or other healthcare provider.

An injectable amniotic fluid-derived preparation may also be injected into a tissue bed in a minimally invasive non-surgical setting. A syringe containing a quantity of the amniotic fluid-derived preparation is fitted with a hypodermic needle of suitable size for the intended application. The needle is directed to the target tissue bed using visualization and palpation of external landmarks by the provider. Placement of the needle within the target tissue space or tissue may, in some embodiments, be facilitated with fluoroscopy or other non-invasive and minimally invasive imaging modalities. Some example uses of the amniotic fluid-derived preparation include intra-articular injection for treatment of injured ligaments, cartilage, and bone; intra-capsular injection of tendon injuries, synovitis, tenosynovitis, and other inflammatory joint conditions; intra-thecal injection for treatment of spinal cord and brain injuries, aseptic meningitis, and other central neurological infections and inflammatory conditions; and other minimally invasive non-surgical applications.

In all of these and other applications, there is strong evidence that the presence active biomolecules in the amniotic fluid-derived preparation improves healing across a broad range of tissue types, locations within the body, and applications. Reporting of clinical results may eventually lead to the use of amniotic fluid-derived preparations as a standard therapy and possibly even a best practice for the treatment of a variety of conditions. Such reporting requires continued laboratory experimentation and human clinical trials to generate additional data for review and interpretation in light of currently available practices and results therefrom. Meaningful interpretation of these data, however, depends on reproducibility. Reproducibility requires standardization of materials and techniques. Standardization in this area should include a cell count per volume and the biologic activity of one or more specific proteins or other biologically active molecules present in the amniotic fluid-derived preparation. In AF from individual donors, substantial differences in both the absolute amount and biologic activity per unit volume of proteins and other biologically active molecules in the final preparation will exist based upon the gestational age at collection, other material and fetal factors, and preparation methods used.

Preparation and sterilization of an amniotic fluid-derived preparation for later use typically includes packaging, sterilization, lyophilization, and storage. Lyophilization discourages bacterial growth and helps maintain sterility during storage. Lyophilization additionally facilitates standardization of the final amniotic fluid-derived preparation in terms of biologic activity per unit volume of the amniotic fluid-derived preparation under standardized parameters. Lyophilization may be accomplished by freezing under controlled conditions to minimize water-ice crystal formation and cellular disruption in products wherein preservation of stem cell viability is desired. Although some viable stem cells are preserved by lyophilization under controlled conditions, other stem cells die during processing. Sterilization by heat or radiation destroys the cellular components of AF, including stem cells. Thermal or irradiative sterilization methods may also denature proteins and alter or destroy other large biologically active molecules. Some amniotic fluid-derived preparations partially reconstitute the concentrated cellular component using a balanced electrolyte tissue preservative solution prior to packaging and storage.

What is lacking in the prior art, therefore, are methods of forming an amniotic fluid-derived preparation incorporating an effective concentration of cellular and biomolecular products from an individual donor within the largest possible pool of volunteer donors with a standardized biological activity, packaged and stored to preserve cellular viability and biological activity of the preparation.

Embodiments of this invention address these and other fundamental requirements of forming an amniotic fluid-derived preparation—providing high concentrations of beneficial biomolecules and viable cells in a standardized preparation with reproducible biologic effects which are preserved throughout packaging, frozen storage, and thawing; essentially no feto-maternal antigenic material, and minimal waste of available donor AF. Disclosed methods of forming an amniotic fluid-derived preparation comprise separating AF into its supernatant and cellular components, assaying and standardizing the supernatant, washing and concentrating the cellular component, and then combining the supernatant and the cellular component with an acceptable fluid to preserve cell viability and biologic activity throughout packaging, freezing, and storage.

Disclosed are methods of forming an amniotic fluid-derived preparation comprising separating a donor amniotic fluid into a supernatant and a cellular component, then combining the supernatant, the cellular component, and a fluid into an amniotic fluid-derived preparation. Some embodiments of the invention comprise additional methods and steps selecting, collecting, processing, standardizing the biologic activity, standardizing the cell count, lyophilizing an acellular derivative, and packaging the product for storage. Amniotic fluid-derived preparations formed by these methods are used by medical providers as an injectable fluid or non-injectable gel preparation, either by intraoperative application or injection, non-operative percutaneous injection, or direct application to injured, ischemic, infected, or otherwise damaged tissue. The amniotic fluid-derived preparations are also used by laboratory researchers as a reproducible source of standardized material for basic science research of the effects of AF preparations on healthy, diseased, and damaged tissue in the field of regenerative medicine, orthopedics, neurology, neurosurgery, gynecologic surgery, and in other clinical, basic medical science, and related scientific disciplines. The use of a reconstituted amniotic fluid-derived preparation comprising biocompatible fluids such as an isotonically balanced buffered electrolyte solution and/or a cryopreservative maximizes delivery of a wide range of regenerative and similarly beneficial biologic substances within a non-antigenic liquid or gel preparation to the treatment site.

Methods of forming amniotic fluid-derived preparations, therefore, comprise steps to separate a donor AF into a supernatant and a cellular component. Following separation, desirable proteins in the supernatant are selected, isolated, concentrated, and quantified with respect to biologic activity. Additionally, desired cells within the cellular component are selected, isolated, washed, and quantified with respect to viability. The concentrated proteins and cells are then combined with a biologically compatible fluid to form an amniotic fluid-derived preparation.

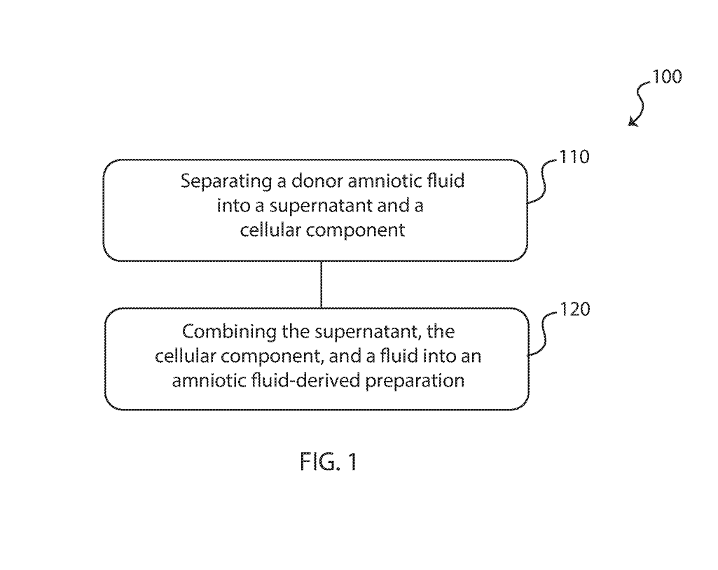

FIG. 1 is a schematic representation of a method 100 of forming an amniotic fluid preparation. Method 100 comprises a separating step 110 and a combining step 120, as shown by FIG. 1.

Separating step 110 comprises separating a donor AF into a supernatant and a cellular component. As mentioned herein above, separating step 100 is performed, in some embodiments, by centrifugation. In some embodiments, separating step 100 is performed by filtration, including ultrafiltration.

In some embodiments, separating step 100 comprises centrifugation. In a non-limiting example embodiment, a donor AF is pipetted into a plurality of fifty milliliter (50 ml) centrifuge tubes. The tubes are sealed and placed in a commercially available centrifuge, such as the Megafuge 8R benchtop centrifuge manufactured by Thermo Scientific, (81 Wyman Street, Waltham, Mass.) and centrifuged at an RCF of 400 g for ten (10) minutes. In some embodiments, the donor AF is centrifuged at 500 g for ten (10) minutes. Centrifugation results in a compacted cellular “pellet” at the bottom of each tube covered by a liquid supernatant that is essentially cell free. The cellular pellet is sufficiently compacted that removal of the supernatant, such as by pipetting or decanting, does not disrupt the pellet and introduces cells back into the supernatant. The aforementioned RCF and duration in this embodiment are by way of example only, alternative RCFs and durations are used in some embodiments. A lower RCF generally requires a longer duration; conversely, a higher RCF can create a compacted pellet following a shorter duration. To separate essentially all intact cells from the AF into a sufficiently compacted pellet, a minimum RCF of about 250 g for a minimum duration of about three (3) minutes comprises separating step 110. Other non-limiting examples include centrifuge speeds from 250 g to 1000 g for a duration from three (3) and fifteen (15) minutes. The choice of speed and duration of AF centrifugation will depend upon factors such as the mechanical fragility characteristics of specific cells retained as viable cells, proteins, and other large molecule substances to be preserved for use in the amniotic fluid-derived preparation.

In some embodiments, a donor AF is centrifuged at an RCF of about 100 g, about 200 g, about 250 g, about 300 g, about 400 g, about 500 g, about 600 g, about 700 g, about 800 g, about 900 g, about 1000 g, about 1250 g, about 1500 g, about 2000 g, between about 100 g and 250 g, between about 250 g and 500 g, between about 500 g and 1000 g, between about 1000 g and about 1500 g, or between about 1500 g and 2000 g. In some embodiments, a donor AF is centrifuged at any of the foregoing RCF values or ranges of RCF values for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, between about 1 minute and 2 minutes, between about 1 minute and 3 minutes, between about 1 minute and 5 minutes, between about 5 minutes and 10 minutes, between about 5 minutes and about 15 minutes, between about 10 minutes and about 15 minutes, or between about 10 minutes and about 20 minutes. In some embodiments, a donor AF is centrifuged at any of the foregoing RCF values, ranges of RCF values, times, and/or ranges of time, at a temperature of about 4° C., about 16° C., about 20° C., about 22° C., about 25° C., about 27° C., about 30° C., or about 37° C.

In some embodiments, the cellular component is “washed,” following removal of the supernatant. The washing of the cellular component is performed by re-suspending cellular component 104 in a buffer solution, such as PBS, for example, and by re-centrifugation (or alternative separation technique) with removal of the supernatant comprising the buffer solution. In some embodiments, multiple cycles of these washing steps are performed. In some embodiments, cellular component 104 is re-suspended, re-centrifuged, and separated from the resulting supernatant two times. In some embodiments, cellular component 104 is re-suspended, re-centrifuged, and separated from the resulting supernatant greater than two times.

In some embodiments, separating step 110 comprises filtration. In a non-limiting example embodiment, a donor AF is gravity-filtered through a ten (10) micron pore-size filter. The filtrate comprises the supernatant. The cellular component remains on the filter, and is washed free of the filter with an appropriate aqueous buffer solution, such as phosphate buffered saline (“PBS”). In some embodiments, the donor AF is tangentially passed across the filter medium to reduce clogging of the filter pores.

In some embodiments, separating step 110 comprises a separation technique other than centrifugation. For example, another technique may be hydrocyclone separation, ultrasonic separation, dielectrophoretic separation, gravitational sedimentation, and the like.

Combining step 120 comprises adding a fluid to the cellular component and the supernatant, to form the amniotic fluid-derived preparation, as shown in FIG. 1. In some embodiments, the fluid comprises an aqueous buffered isotonic solution, such as PBS as a non-limiting example. In some embodiments, the fluid is a combination of a buffered isotonic solution, a cryoprotectant, and any other non-cytotoxic fluid. In some embodiments, the fluid comprises a buffered isotonic solution. An additional non-limiting example of a buffered isotonic solution is “Plasma-Lyte A,” manufactured by Baxter International, Inc., Deerfield, Ill. In some embodiments, the fluid comprises a cryopreservative, such as CryoStor CS-10, a 10% solution of dimethylsulfoxide (“DMSO”) manufactured by BioLife Solutions, Inc., Bothel, Wash., for example. In some embodiments, the fluid comprises a 5% solution of DMSO. These examples are not meant to be limiting, other examples of non-cytotoxic buffering and cryoprotectant fluids are used, in some embodiments.

In some embodiments, the volume of fluid 130 relative to the volume of the supernatant and the volume of cellular component is calculated based upon a desired end-biologic activity in the amniotic fluid-derived preparation being formed. In some embodiments, the volume of the fluid is based upon a desired end-viable cell count in the amniotic fluid-derived preparation being formed. Following combination of the supernatant and the cellular component with the fluid, final concentrations of viable cells and protein activity per unit volume of the amniotic fluid-derived preparation are calculated, in some embodiments. In some embodiments, the biologic activity is assayed and additional fluid in an amount calculated to dilute the assayed biologic activity to a desired level is added. In some embodiments, the viable cell count is re-counted and additional fluid in an amount calculated to dilute the re-counted cell count to a desired level is added.

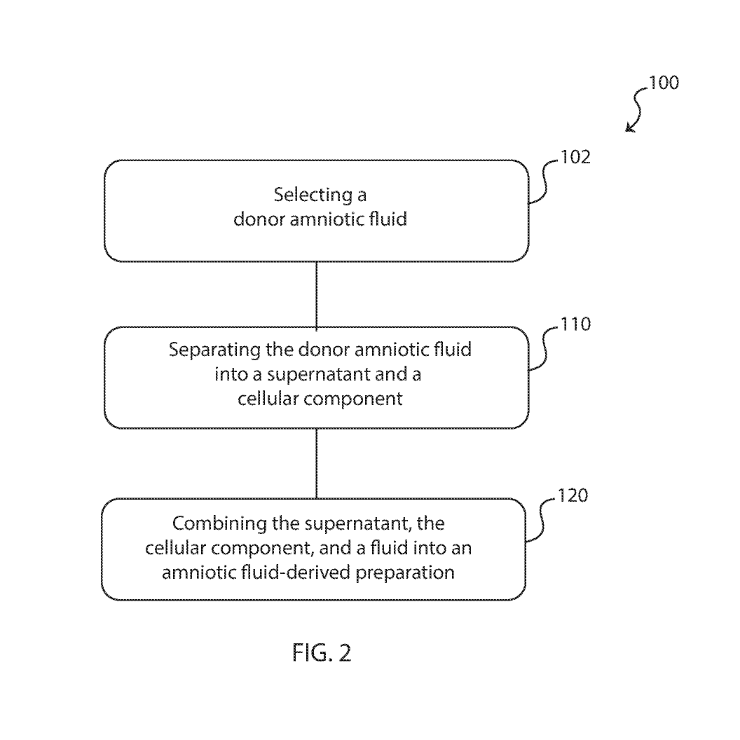

FIG. 2 is a schematic representation of a further embodiment of method 100 of forming an amniotic fluid preparation. The embodiment of method 100 shown in FIG. 2 comprises a selecting step 102, the separating step 110, and the combining step 120 of method 100.

Selecting step 102 comprises selecting a donor amniotic fluid. Amniotic fluid for amniotic fluid-derived preparations is potentially available in substantial quantities from a pool of donors. There are just under 4 million births per year in the United States, making up a pool of potential AF donors. From this pool, AF can be selected and made available from a suitably screened subpopulation.

In some embodiments, the donor AF is selected prior to separating step 110 and combining step 120. A volunteer human donor is selected and screened, in some embodiments. Accepting AF from volunteer donors and excluding any non-volunteer and paid donors from the donor pool is consistent with internationally well-established tissue donation protocols and reduces the risk that any infectious agents present in the donor will be transmitted to the recipient of an amniotic fluid-derived preparation, resulting in an infection in the recipient. In some embodiments, therefore, selecting step 102 comprises screening of potential volunteer donors. This screening includes a subjective component and an objective component, in some embodiments. Subjective screening comprises obtaining a comprehensive past medical and social history. This history may be obtained by the potential volunteer donor responding to a questionnaire, providing responses to a healthcare provider interview, or a combination of a questionnaire and a provider interview.

Objective screening, in some embodiments, comprises complete blood count, liver and metabolic profile, and serologic testing for HBV, HCV, HIV. In some embodiments, serological testing for other infectious agents is performed. In some embodiments, a donor AF may be free or substantially free of HBV, HCV, HIV, and/or other infectious agents. In some embodiments a donor AF may be selected for being free or substantially free of HBV, HCV, HIV, and/or other infectious agents.

AF from acceptable donors may additionally be excluded in selecting step 102 by perinatal observations and events. Clinical or laboratory evidence of active maternal or fetal infections around the time of delivery, the most severe example manifest by chorioamnionitis, precludes the use of AF. Meconium staining of the AF and/or the fetal membranes, although usually not indicative of infection, also may eliminate the individual from the donor pool. Finally, and most commonly, contamination of the placental membranes with a large quantity of maternal blood, feces, or other perinatal sources of gross bacterial or tissue contamination precludes use of the AF.

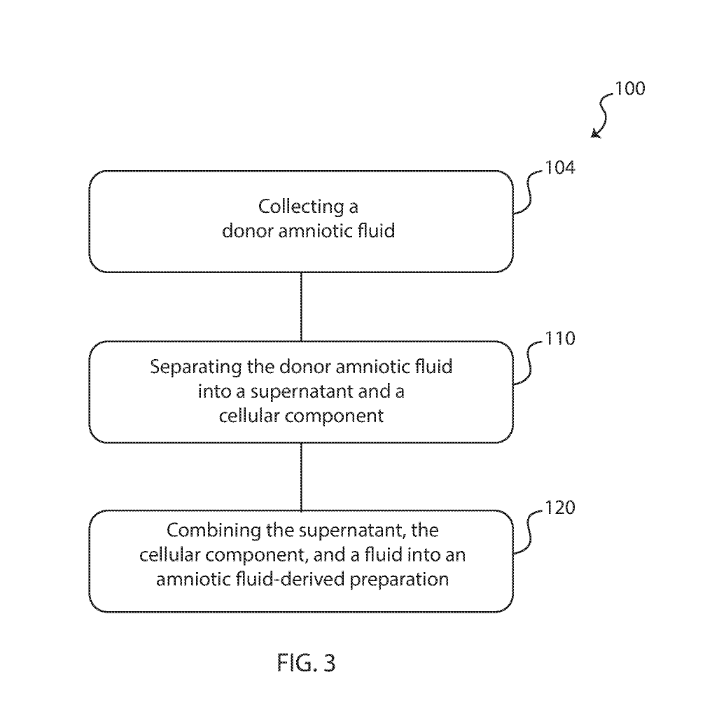

FIG. 3 is a schematic representation of a further embodiment of method 100 of forming an amniotic fluid preparation, comprising a collecting step 104, separating step 110, and combining step 120.

Collecting step 104 comprises collecting a donor AF. The donor AF is collected using a variety of methods in a variety of settings. For example, in some embodiments, the donor AF is collected during a surgical Cesarean section delivery of the infant. In some embodiments, donor AF is collected from a non-human donor.

It is generally most practical to collect the donor AF during delivery by Cesarean section. The use of a Cesarean-obtained donor amniotic fluid 105 to prepare amniotic fluid-derived preparation 100 is preferable in some embodiments because donor amniotic fluid 105 collected by Cesarean section is obtained directly from the uterine cavity without contacting a non-sterile structure; i.e., the vagina; and packaged under strict sterile technique in the operating room with essentially no microbial contamination. In some embodiments, the donor AF is collected into a sterile suction canister liner by the surgeon-obstetrician by nicking the amniotic membrane and inserting the suction tip into the placental sac under direct vision so as to prevent injury to the infant. Following suction-collection of the donor AF, which is completed within five to ten seconds following trans-uterine insertion of the suction tip into the amniotic sac, the baby is immediately delivered by the surgeon-obstetrician.

Despite the preference for a Cesarean-collected donor AF, vaginally collected donor AF is utilized in some embodiments to increase the pool of potential donors. A situation wherein this is practical, in some embodiments, is when trans-vaginal rupture of fetal membranes is indicated to initiate or promote the progression of labor. Similar sterile collection and handling practices as discussed herein above are utilized, although the donor AF is collected through a sterile suction cannula placed in the vagina in immediate proximity to the cervix prior to rupturing the fetal membranes with an amnion hook or similar instrument. Great care must be afforded the vaginally-collected donor AF to prevent microbial contamination. Vaginally-collected AF is not an acceptable donor AF if there is fecal, blood, or other grossly visible contamination noted in the AF or in proximity to the vagina at the time of collection. Neither a vaginally-collected donor AF nor a Cesarean-collected donor AF is acceptable to form the amniotic fluid-derived preparation if meconium is present in the AF or if there is any visible meconium discoloration or staining of the AF.

In some embodiments, the donor AF comprises AF collected from a non-human donor animal. A lack of expression of HLA-1 and HLA-D related (“HLA-DR”) epitopes makes cross-species use of amniotic fluid-derived preparations possible. In some embodiments, the amniotic fluid-derived preparation comprises donor AF from a non-human donor which is completely de-cellularized by processing prior to combining step 120. For example, in some embodiments, AF from a non-human donor animal is placed in a centrifuge at 400 g for ten (10) minutes, rendering the resulting supernatant free of cells and cellular debris. In a second non-limiting example, the AF from a non-human donor animal is filtered through a filter with a 0.22 micrometer pore size, wherein all cells and cellular debris are removed from donor AF.

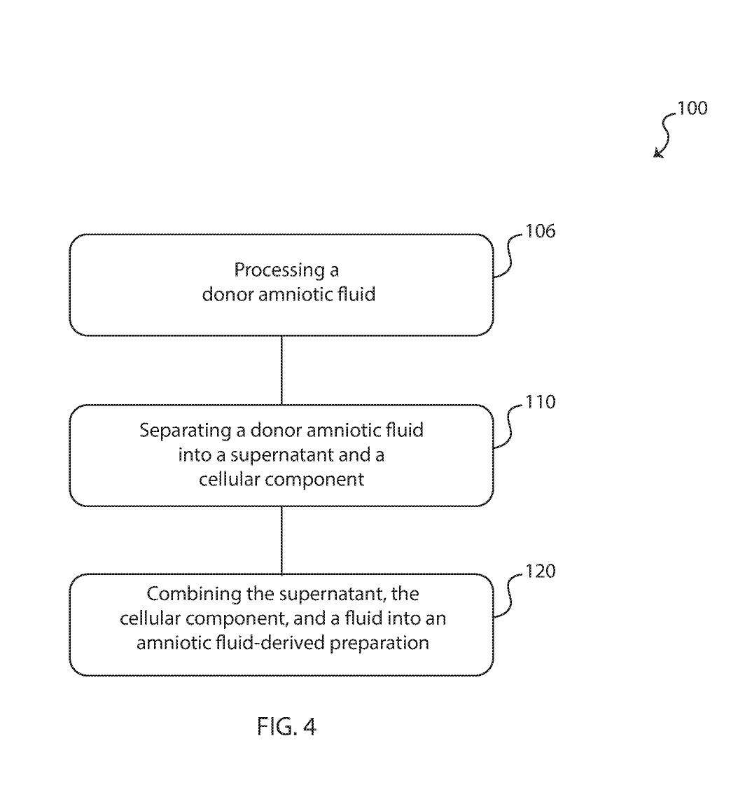

FIG. 4 is a schematic representation of is a schematic representation of a further embodiment of method 100 of forming an amniotic fluid preparation. FIG. 4 shows method 100 comprising a processing step 106, separating step 110, and combining step 120.

During processing step 106, operating room personnel familiar with sterile technique and tissue handling perform all steps necessary to prepare the donor AF for initial processing. In some embodiments, the sterile container containing the donor AF collected in the operating room is then securely closed and placed in a donor tissue specimen bag. In some embodiments, this first bag is then placed within a second bag, which is then sealed, labeled, and taken from the operating room for packaging in an insulated ice-bath container. A patient data sheet containing information regarding the maternal donor is placed in the container, and a separate copy of this information is recorded and logged prior to closing the package, in some embodiments. In some embodiments, the packaged specimen container is then immediately transported to a processing facility by staff who rotate on call, such that there is minimal delay following delivery before the donor tissue arrives at the separate facility for processing. In some embodiments, the separate facility is a proprietary processing facility in a separate location from the hospital wherein the donor AF was collected.

Additional procedures comprise processing step 106, in some embodiments. In a non-limiting example, following collection, on-call staff are notified that the donor AF is inbound to the processing facility. The staff respond by preparing required supplies and equipment to immediately receive and process the donor AF. Donor AF is transported in a shipping container and, upon arrival of the donor AF, the staff check the integrity of the shipping container, verifying that a tamper-proof seal is unbroken and a shipping label is intact. Staff then open the shipping container and confirm that the temperature at the surface of the sterile back bearing the donor AF is between about six (6) and about ten (10) degrees Celsius. Staff validate the donor credentials by recording a donor lot number and collection date, the date and time of receipt and validation, and make sure that the donor paperwork matches the donor ID sticker affixed to the tissue bag containing the donor AF. Staff record the time, date, and location where the donor AF is to be stored within the processing facility. Staff spray the outside of the tissue bag holding the donor AF with an alcohol solution and wipe it down with a sterile cloth. At which day and time separating step 110 is to be performed, staff complete processing step 106. Staff remove the tissue bag containing the donor AF and place it in a pass-through refrigerator for removal within the clean processing area. All staff handling the donor AF are to be freshly showered and wearing no lotions, perfumes, or body oils. All staff change from street clothing into pre-packaged scrubs, processing shoes, and gowns in a staff locker room designated for this purpose. Staff then enter a staging area between the staff locker room and the clean processing area, wherein staff don a mask, goggles, hair bonnet, and place two pairs of disposable booties over their shoes. Staff then enter the clean processing room. In the clean processing room, staff confirm that all needed supplies and equipment are available. Necessary equipment, such as a light microscope, a bench centrifuge, a hemocytometer, calculator, Trypan blue, scissors, and sterile towels are properly set up and positioned. At least one staff member stays “clean” but does not scrub, wherein the clean staff assist scrubbed staff by opening packages of sterile instruments and equipment, etc. Additional staff then scrub, don a sterile gown and gloves, and set-up a sterile field by placing a sterile cover on the table and placing a sterile basin, cutting board, forceps, and any other necessary sterile instruments on the sterile cloth-covered table.

Following processing step 106, the donor AF is separated according to separating step 110 and combined with a non-biological fluid according to combining step 120 for form the amniotic fluid-derived preparation.

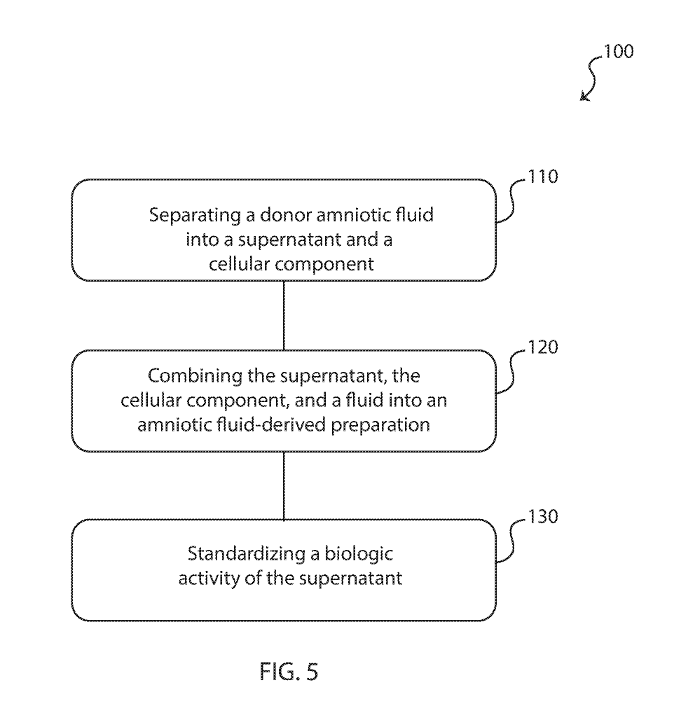

FIG. 5 is a schematic representation of a further embodiment of method 100 of forming an amniotic fluid preparation. FIG. 5 shows method 100 comprising separating step 100, combining step 120, and first standardizing step 130.

First standardizing step 130 comprises standardizing a biologic activity of the supernatant. In some embodiments, first standardizing step 130 is performed on the preparation product following combining step 120, as shown in FIG. 5. This is not meant to be limiting, however. In some embodiments, first standardizing step 130 is performed on the supernatant prior to combining step 120.

Following separating step 110, an AF supernatant is formed. This water-based AF supernatant, in turn, may comprise a large number of distinct substances, including an AF proteome, electrolytes, phospholipids, carbohydrates, and/or urea. The AF proteome is the entire set of products of transcription manifest as proteins and polypeptides within donor AF, and may vary between individual AF donor sources.

First standardizing step 130 is performed using available techniques specific to the protein of interest. The normal human AF proteome comprises over one hundred and thirty (130) separate proteins (Tsangaris, et al. (2006) “The Normal Human Amniotic Fluid Supernatant Proteome” In Vivo 20:279-90.). These individual proteomic proteins comprise products of maternal and fetal transcription, and may vary somewhat depending upon the gestational age of the fetus, the gender of the fetus, and any existing fetal or maternal chromosomal or metabolic abnormalities. Multiple techniques are known in the art to separate, isolate, and concentrate proteins from complex biological fluids, such as the AF supernatant. Such techniques may alter the composition and biologic activity of individual protein constituents of the AF supernatant. In some embodiments, the supernatant may be further processed to comprise any one protein or plurality of proteins in any desired number or combination, depending upon the intended use of the amniotic fluid-derived preparation. In some embodiments, for example, the supernatant is the complete AF supernatant proteome. In some embodiments, a portion of the water and non-protein constituent compounds of the AF supernatant are removed, wherein the overall proteomic concentration is increased using techniques known to those in the art, such as precipitation, for example.

Some non-limiting examples individual proteins of interest are a growth factor, such as a vascular endothelial growth factor, hepatocyte growth factor, angiopoietin, platelet-derived growth factor, and fibroblast growth factor; a signaling ligand, such as monocyte chemoattractant protein-1, stromal cell derived factor-1, and stem cell factor; extracellular tissue remodeling proteins, such as matrix metalloproteinases and tissue inhibitors of matrix metalloproteinases; receptor molecule antagonists, such as interleukin-1; cytokines, such as interleukin-10 and prostaglandin E-2; transcriptional regulators, such as octamer-binding transcription factor 4; and immune regulators, such as transforming growth factor beta.

It is desirable, in some embodiments, to know the concentration or the biologic activity of a protein or other substance of interest in the amniotic fluid-derived preparation following combining step 120. First standardizing step 130 measures the concentration of the protein of interest within the amniotic fluid-derived preparation, in some embodiments. In some embodiments, first standardizing step 130 measures the biologic activity of the protein of interest within the amniotic fluid-derived preparation. Measuring techniques and protocols known in the art are employed to perform first standardizing step 130. For example, in some embodiments, first standardizing step 130 comprises use of an enzyme-linked immunosorbent assay (“ELISA”) to measure the concentration of the protein of interest. In some embodiments, first standardizing step 130 is performed at a clinical laboratory improvement amendment (“CLIA”)-certified laboratory facility. If an ELISA assay shows a quantity of the protein of interest below a designated range of the protein of interest, the supernatant will not be used to create the amniotic fluid-derived preparation. Conversely, if an ELISA assay shows a quantity of the protein of interest above a designated range of the protein of interest for the final amniotic fluid-derived preparation, the concentration of the protein of interest within the combined product is further diluted by adding a quantity of a non-biologic fluid calculated to achieve the desired concentration of the protein of interest, such as the fluid used in combining step 120, to form the amniotic fluid-derived preparation.

In a different example wherein the biologic activity of the protein of interest, expressed in activity units per volume, is measured. For example, the biologic activity of vascular endothelial growth factor (“VEGF”), and other pro-angiogenic factors, can be established with an in vitro tube assembly assay. A tube assembly assay measures the degree to which a population of vascular endothelial cells organize into hollow tubes following exposure to a preparation comprising a regulatory protein, such as vascular endothelial growth factor, which promotes endothelial tube formation. Following preparation of a culture of non-confluent vascular endothelial cells, a small measured quantity of supernatant is added to the culture. The culture is incubated for four (4) to six (6) hours in a compatible culture medium and under standard conditions for cell growth known in the art and then the tube network is microscopically examined, noting the number and length of endothelial cell tubes formed. The concentration of VEGF in the supernatant can be extrapolated from a standard curve comparing concentration per microliter to endothelial cell tube number and tube length. The aforementioned general procedure is by way of example only. Other known procedures using commercially available materials are performed to measure concentration, function, or biologic activity of the protein of interest in the supernatant.

In some embodiments wherein the concentration, function, or biologic activity of the protein of interest is below the end value required, the supernatant is not used to form the amniotic fluid-derived preparation. In some embodiments wherein the concentration, function, or biologic activity of the protein of interest is above the end value required, the concentration of the protein of interested within the combined product is further diluted by adding a quantity of a non-biologic fluid calculated to achieve the desired concentration of the protein of interest, such as the fluid used in combining step 120, to form the amniotic fluid-derived preparation.

In some embodiments, the concentration, function, or biologic activity of the protein of interest is measured in the combined product following combining step 120 to form the amniotic fluid-derived preparation. In some embodiments, the concentration, function, or biologic activity of the protein of interest is measured in the supernatant prior to combining step 120.

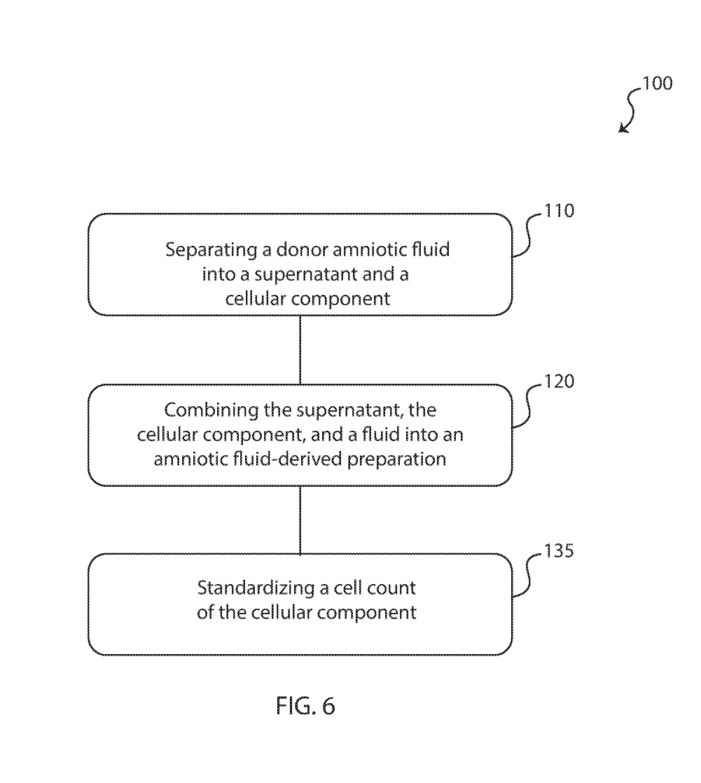

FIG. 6 a schematic representation of a further embodiment of method 100 of forming an amniotic fluid preparation. FIG. 6 shows method 100 comprising separating step 110, combining step 120, and second standardizing step 135. Second standardizing step 135 comprises standardizing a cell count of the cellular component. When collected and prior to processing, the donor AF comprises many different cell types. These constituent cell types may be generally divided into two groups: 1) progenitor cells; and 2) differentiated cells. The progenitor cell component may be further divided into a pluripotent cell group and a committed cell group. Pluripotent cells retain the ability to differentiate into any germ line; i.e. endodermal, mesodermal, or ectodermal-derived tissues. Committed progenitor cells will differentiate into defined germ cell lines or organ-specific cell types. Various morphological characteristics are used to distinguish between progenitor cells and differentiated cells. A suspension of the cellular component in an aqueous buffer fluid is prepared and placed on a counting grid, such as a hemocytometer for example. Cells bearing a particular characteristic distinctive for a progenitor cell or a differentiated cell of a particular type are noted and counted on the grid using standard techniques known in the art and the concentration of the desired cell in the suspension is noted. From this concentration, a quantity of fluid, such as the fluid added in combining step 120, is used to adjust the concentration such that the amniotic fluid preparation bears the required cell count of the cell type of interest from the cellular component.

In some embodiments, the cell count of the cellular component is a total cell count of all cell types of the cellular component. In some embodiments, the cell count of the cellular component is a total viable cell count of the cellular component. Counting viable cells is accomplished, in some embodiments, with a standard trypan blue exclusion assay. For example, in some embodiments, the cell density of the cell type of interest is determined by using a hemocytometer. One hundred microliters of a 0.4% solution of trypan blue in a buffered isotonic saline solution with a pH of about 7.2 to a pH of about 7.3, such as PBS, is added to one milliliter of suspended cells. The trypan blue-cell suspension mixture is immediately placed in a hemocytometer and examined using light microscopy. Viable cells not stained with Trypan blue are counted and the concentration of viable cells within the cellular suspension is counted.

In some embodiments, the final cell concentration or the final viable cell concentration is about 10,000 cells per milliliter, about 50,000 cells per milliliter, about 100,000 cells per milliliter, about 250,000 cells per milliliter, about 500,000 cells per milliliter, about 1×10⁶ cells per milliliter, about 2×10⁶ cells per milliliter, about 5×10⁶ cells per milliliter, about 7.5×10⁶cells per milliliter, about 1×10⁷cells per milliliter, between about 10,000 cells per milliliter and about 100,000 cells per milliliter, between about 100,000 cells per milliliter and about 1×10⁶ cells per milliliter, between about 1×10⁶ cells per milliliter and about 2×10⁶ cells per milliliter, between about 2×10⁶ cells per milliliter and about 5×10⁶ cells per milliliter, or between about 5×10⁶ cells per milliliter and about 1×10⁷ cells per milliliter. In some embodiments, the final cell concentration or the final viable cell concentration is between zero (0) and 1.5 million cells per milliliter. In some embodiments, the final cell concentration or the final viable cell concentration is greater than 2.5 million cells per milliliter. In some embodiments, the final cell concentration or the final viable cell concentration is between 1.5 and 2.5 million cells per milliliter. In some embodiments, the final cell concentration or the final viable cell concentration is greater than two (2) million cells per milliliter.

In some embodiments, second standardizing step 135 is performed on the combined product following combining step 120, as shown in FIG. 6. This is by way of example and not meant to be limiting; in some embodiments, second standardizing step 135 is performed on the cellular component prior to combining step 120.

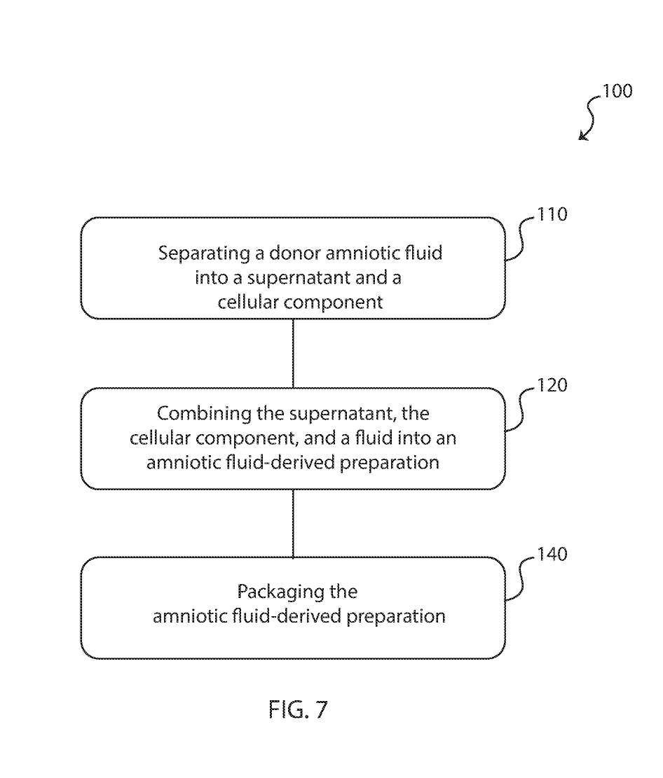

FIG. 7 is a schematic representation of a further embodiment of method 100 of forming an amniotic fluid preparation. FIG. 7 shows method 100 comprising separating step 110, combining step 120, and packaging step 140.

Packaging step 140, in some embodiments, comprises packing the amniotic fluid-derived preparation. In some embodiments, packaging step 140 comprises freezing. In some embodiments, packaging step 140 comprises controlled-rate freezing. For example, using a controlled rate freezer, such as a Thermo Scientific Controlled Rate Freezer, Model number 7454 on cycle 1-2 (−2° C. per minute) bringing the product down to a temperature of −80° C. In some embodiments, for example, the amniotic fluid-derived preparation is sealed in sterile packaging vials, frozen at a temperature of minus eighty degrees Celsius (−80° C.), and held at this temperature until shipping.

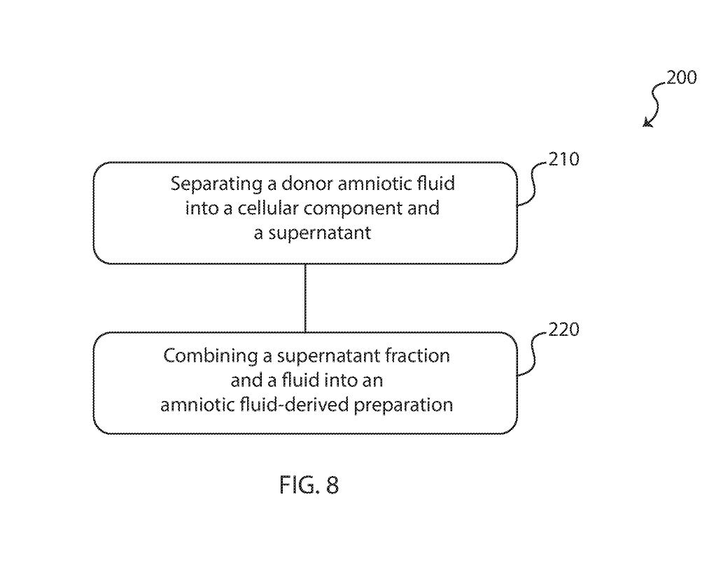

FIG. 8 is a schematic representation of a method 200 of forming an amniotic fluid preparation. Method 200, as shown in FIG. 8, comprises a separating step 210 and a combining step 220. Separating step 210 comprises separating a donor amniotic fluid into a cellular component and a supernatant. Combining step 220 comprises combining a supernatant fraction and a fluid into an amniotic fluid-derived preparation. Of note separating step 210 of method 200 and separating step 110 of method 100 are similar steps. The combining step 220 of method 200 distinguishes method 200 from method 100, in that combining step 220 of method 200 does not combine the cellular component into the formed amniotic fluid-derived preparation. Wherein amniotic fluid-derived preparation is an acellular solution, the preparation can be stored considerably longer than a cellular solution wherein viability of the cellular component is desired by the user. In some embodiments, therefore, the amniotic fluid-derived preparation does not comprise a cellular component.

Following separating step 210 of method 200, the supernatant is combined with a non-biologic fluid, such as PBS, other aqueous-based buffer solution, a cryoprotectant, or another fluid, some examples of which that are disclosed herein above. In some embodiments, the final cell-free amniotic fluid-derived preparation is standardized with respect to a protein concentration, protein function, or protein biologic activity as disclosed herein above.

In some embodiments, protein concentration techniques, such as precipitation or dialysis, are employed to increase the concentration of proteins and other substances present in the supernatant prior to combining step 220 of method 200. For example, in some embodiments precipitation of proteins with ammonium sulfate is performed. Ammonium sulfate in solution causes reversible precipitation of most proteins, and does not cause protein denaturation. The crystalline solid is added to the supernatant slowly, in multiple aliquots, with stirring until the ammonium sulfate concentration reaches about thirty percent (30%) by weight. This is by example only; the percent of ammonium sulfate by weight may be as low as about twenty percent (20%) or as high as seventy percent (70%), depending upon the solubility characteristics of the protein(s) which are to be precipitated for concentration in the supernatant fraction. For example, in some embodiments, the percent of ammonium sulfate by weight may be about 20%, about 30%, about 40%, about 50%, about 60%, or about 70%. Ammonium sulfate is added at room temperature and stirring, whether continuous or intermittent, continues for about fifteen (15) minutes. The suspension is then centrifuged. The resulting second supernatant is removed and the protein precipitate pellet, which comprises the supernatant fraction, is combined with a fluid to form the amniotic fluid-derived preparation in combining step 220.

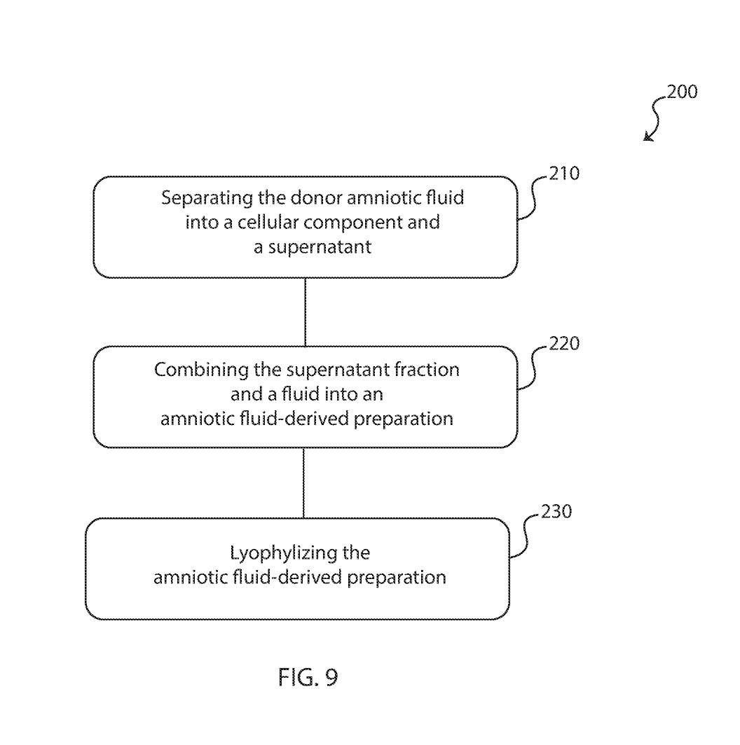

FIG. 9 is a schematic representation of a further embodiment of method 200 of forming an amniotic fluid preparation. In the embodiments shown in FIG. 9, and some other embodiments, method 200 further comprises lyophilizing step 230. Lyophilizing step 230, in some embodiments, comprises lyophilizing the amniotic fluid-derived preparation. Lyophilizing step 230 is performed wherein amniotic fluid-derived preparation need not comprise viable cells, as lyophilization is known to kill essentially all living mammalian cells. Storage of proteins in an aqueous medium for lengthy periods decreases the functionality of the stored proteins. Water, over time, induces aggregation and denaturation, as well as chemical degradation by hydrolysis and deamidation. Lyophilization, however, stabilizes the stored protein and allows the constituent proteins of the amniotic fluid-derived preparation to remain functional after reconstitution following lengthy storage at −80° C.

Lyophilizing step 230 is performed, in some embodiments, using a commercially available vacuum freezer with a controlled rate cycle. In some embodiments, a protein-stabilizing excipient compound, such as sucrose, other disaccharides, or trehalose, for example, is added prior to lyophilization.

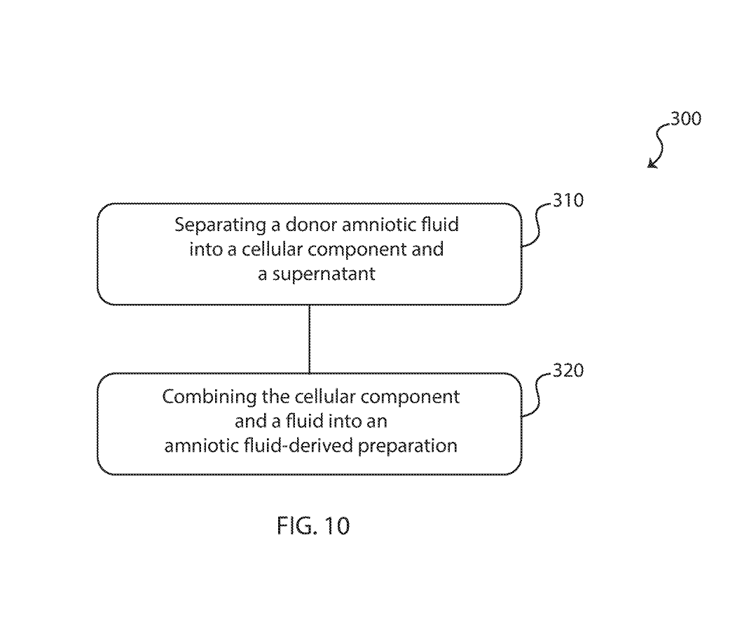

FIG. 10 is a schematic representation of a method 300 of forming an amniotic fluid preparation. Method 300 comprises a separating step 310 and a combining step 320. Separating step 310 comprises separating a donor amniotic fluid into a cellular component and a supernatant. Combining step 320 comprises combining the cellular component and a fluid into an amniotic fluid-derived preparation. Method 300 is distinguished from method 100 and method 200 by combining step 320, wherein only the cellular component, and not the supernatant, is combined with the fluid to form an amniotic-derived fluid preparation. In some embodiments, the cellular component is “washed” by suspension in a small quantity of the fluid, or other biologically compatible buffered isotonic aqueous fluid, and re-centrifuged. The new supernatant is removed and the cycle is repeated one or more times, in some embodiments. This washing process serves to remove extracellular proteins, nucleic acids, and other extracellular elements from the cellular component.

In some embodiments, the entire donor AF cellular component is combined with the fluid in combining step 320. In some embodiments, certain cell types are removed using various techniques available and mentioned herein below, by example. In some embodiments, a single cell type or cell family, based upon specific cell surface receptor makers and other immunological markers, is removed from the remainder of the separated donor AF (following separating step 310) and combined with the fluid in combining step 320 to form the amniotic fluid-derived preparation.

The cellular component, in some embodiments, is separated into groups of constituent cell subtypes which are isolated using various techniques known in the art and concentrated in the amniotic fluid-derivative preparation. Some non-limiting examples of these cell types comprising the cellular component which is combined with the fluid in combining step 320 include cells bearing surface receptors identifying the cell as a mesenchymal stem cell, a progenitor cell, or an epithelial cell, such as a cell expressing surface receptor CD44, a cell expressing surface receptor CD29, a cell expressing surface receptor CD49e, a cell expressing surface receptor CD54, a cell expressing surface receptor CD44, a cell expressing surface receptor CD326, a cell expressing surface receptor CD166, a cell expressing surface receptor CD271, a cell expressing surface receptor CD349, and/or a cell expressing surface receptor CD140b.

In some embodiments, the cellular component is substantially depleted of epithelial cells, mesenchymal cells, or of any of the aforementioned cells bearing cell surface receptors identified by non-limiting example in the preceding paragraph.

Some non-limiting examples of cell separation techniques used to select for specific cell types and cell families in the cellular component include density-gradient centrifugation, magnet-activated cell sorting (“MACS”), polymer-bound monoclonal antibodies, other antibody-based techniques, microfluidic techniques, and the like.

In some embodiments, density-gradient centrifugation within a sucrose solution or a colloidal silica suspension, such as Percoll®, is employed to separate the heterogeneous cell populations comprising the cellular component into a number of subpopulations based upon the buoyant density of the subtype. During centrifugation, cells may “band” on the gradient in levels corresponding to the relative buoyant density of each subpopulation. The region containing the desired subpopulation to comprise the cellular component is removed from the banded supernatant. Conversely, a region not comprising the desired cellular component is removed, in some embodiments. A region not comprising the cellular component may be a region comprising dead cells.

In some embodiments, viability testing of the cellular component is conducted to remove non-viable cells from the cellular component. In some embodiments, viability testing comprises a standard Trypan blue exclusion technique, known in the art and discussed herein above.

In some embodiments, magnetized polymer microbeads, such as Dynabeads®, are reversibly coupled to a specific cell type by a monoclonal cell-surface receptor antibody. In some embodiments, amniotic epithelial cells comprising cell surface receptors CD326, are separated and removed from the cellular component utilizing magnetized polymer microbeads coupled to monoclonal antibodies to the CD326.

In some embodiments, the cellular component comprises an epithelial stem cell. In some embodiments, the cellular component comprises a mesenchymal stem cell. In some embodiments, the cellular component is substantially depleted of mesenchymal cells. In some embodiments, the cellular component is substantially depleted of epithelial cells.

In some embodiments, combining step 320 comprises diluting the cellular component with the fluid, or some other suitable isotonic buffer solution. In some embodiments, a cell count per unit volume of the suitable buffer solution is determined using techniques discussed herein above and the cellular component is further diluted to a desired cell count per unit volume.

The completed amniotic fluid-derived preparation is sealed in pre-sterilized packaging vials and frozen for storage at minus eighty (−80) degrees Celsius, in some embodiments.

Methods of forming an amniotic fluid-derived preparation are disclosed and discussed. The disclosed methods are practical to perform and result in amniotic fluid-derived preparations that retain protein bioactivity and cellular viability, are quantified with respect to protein and cellular components, and are packaged and stored for convenient and practical use by a clinician or researcher.

### Incorporation by Reference

The entire disclosure of each of the patent documents and scientific articles cited herein is incorporated by reference for all purposes.

### Equivalents

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. In drawings wherein method steps are represented, the order of steps presented in a flow chart is by example only and not intended to create a limitation that the steps be performed in the order represented, or in any specific order, unless otherwise stated otherwise herein. Many additional modifications and variations are possible in light of the teachings above, and are intended to fall within the scope of the appended claims. The disclosure can be embodied in other specific forms without departing from the essential characteristics thereof. The scope of the disclosure is indicated by the appended claims rather than by the foregoing description, and all changes that come within the meaning and range of equivalency of the claims are intended to be embraced therein.

What is claimed is:

1. A method of forming an amniotic fluid-derived preparation, the method comprising the steps of:
separating a donor amniotic fluid into a supernatant and a cellular component; and
combining the supernatant, the cellular component, and a fluid to form an amniotic fluid-derived preparation.

2. The method of claim 1, wherein the separating step comprises centrifuging the donor amniotic fluid.

3. The method of claim 1, wherein the separating step comprises filtering the donor amniotic fluid.

4. The method of claim 1, wherein the fluid comprises a cryopreservative.

5. The method of claim 1, wherein the fluid comprises a buffered electrolyte solution.

6. The method of claim 1, the method further comprising a step of selecting the donor amniotic fluid.

7. The method of claim 6, wherein the selecting step comprises determining a risk of a transmittable infectious disease in the donor amniotic fluid.

8. The method of claim 1, the method further comprising a step of collecting the donor amniotic fluid.

9. The method of claim 8, wherein the collecting step comprises removing the donor amniotic fluid from the uterus of a pregnant individual by Cesarean section.

10. The method of claim 8, wherein the collecting step comprises trans-vaginally removing the donor amniotic fluid.

11. The method of claim 1, the method further comprising a step of processing the donor amniotic fluid.

12. The method of claim 1, the method further comprising a step of standardizing a biologic activity of the supernatant.

13. The method of claim 1, the method further comprising a step standardizing a cell count of the cellular component.

14. The method of claim 1, the method further comprising a step of packaging the amniotic fluid-derived preparation.

15. The method of claim 1, wherein the supernatant is acellular.

16. A method of forming an amniotic fluid-derived preparation, the method comprising the steps of:
separating a donor amniotic fluid into a supernatant and a cellular component; and
combining the supernatant and a fluid to form an amniotic fluid-derived preparation.

17. The method of claim 16, the method further comprising a step of lyophilizing the amniotic fluid-derived preparation.

18. The method of claim 16, wherein the supernatant is acellular.
